(19) United States
(12) Patent Application Publication
(10) Pub. No.: US 2025/0114638 A1
(43) Pub. Date: Apr. 10, 2025

(54) RADIOTHERAPY SYSTEM, METHOD FOR DELIVERING NEUTRON ACCEPTOR, AND METHOD FOR TREATING OR DIAGNOSING CANCER

(71) Applicant: National Tsing Hua University, Hsinchu (TW)

(72) Inventors: Chi-Shuo Chen, Hsinchu City (TW); Chun-Ting Lin, Taoyuan City (TW); Fang-Hsin Chen, Taipei City (TW); Chen-En Chiang, Kaohsiung City (TW); Jia-Jun Liu, Kaohsiung City (TW); Chih-Tung Liu, New Taipei City (TW); Jui-Hsun Chang, New Taipei City (TW); Jun-Chen Wu, Taichung City (TW); Po-Chun Huang, New Taipei City (TW); Hui-Ling Lin, Taoyuan City (TW); Ya-Fang Liu, Hualien County (TW)

(73) Assignee: National Tsing Hua University, Hsinchu (TW)

(21) Appl. No.: 18/882,784

(22) Filed: Sep. 12, 2024

Related U.S. Application Data
(60) Provisional application No. 63/537,852, filed on Sep. 12, 2023.

(51) Int. Cl. A61N 5/10

(52) U.S. Cl. CPC A61N 5/1001; A61N 2005/1019; A61N 2005/109

(57) ABSTRACT

A radiotherapy system includes a neutron acceptor, an aerosolization device for aerosolizing the neutron acceptor, and an energy beam generator. A method for treating cancer, including: administering to a subject in need thereof an effective amount of an aerosolized neutron acceptor; and irradiating the subject with neutrons. A method for diagnosing cancer, including: administering to a subject in need thereof an effective amount of an aerosolized radioactive agent; and receiving an energy beam signal from the subject. A method for delivering a neutron receptor to a subject during radiation therapy, including aerosolizing the neutron receptor and administering to the subject an effective amount of the neutron receptor.

### CROSS REFERENCE TO RELATED APPLICATION

This application claims the benefit of U.S. Provisional Application No. 63/537,852, filed on Sep. 12, 2023. The content of the application is incorporated herein by reference.

### BACKGROUND OF THE INVENTION

### 1. Field of the Invention

The present disclosure relates to radiotherapy, and more particularly to neutron capture therapy.

### 2. Description of the Prior Art

Boron neutron capture therapy (BNCT) is a targeted radiation therapy that exploits the high absorption characteristics of tumor cells for boron-10 drugs. Tumor cells that absorb boron-10 drugs have a greater propensity to capture neutrons and undergo high-energy nuclear fission reactions (¹⁰B(n, α)⁷Li), releasing high-linear energy transfer particles: alpha particles (⁴He), lithium particles (⁷Li), and approximately 2.4 MeV of energy. The travel distance of these high-linear energy transfer particles is approximately 10 μm, which is roughly the diameter of a cell. Consequently, they can directly inflict lethal damage to the DNA of tumor cells. On the contrary, other tissues in the body composed of basic elements such as carbon, hydrogen, oxygen, and nitrogen do not easily capture neutrons. This property enables BNCT to create a substantial dose gradient between tumor cells and normal cells. It is advantageous for treating locally recurrent tumors that are difficult to irradiate or have unclear boundaries with normal tissues, thereby minimizing harm to healthy tissues and providing patients with treatment in a less detrimental manner.

It is crucial for boron-10 drugs to accumulate highly and selectively deliver to tumor cells to enhance the therapeutic efficacy of BNCT and avoid damage to neighboring healthy cells. According to the current clinical standards of BNCT, average tumour-to-normal tissue ratio (T/N ratio) of the boron-10 drugs needs to be greater than 2.5. However, the boron-10 delivery agents are usually administered intravenously to patients during clinical BNCT treatment, and these low molecular weight compounds are easily eliminated from tumor cells and blood, which results unsatisfactory T/N ratio.

Therefore, there remains an urgent and unmet need in the art for an approach to enhancing T/N ratio of boron reagents and expanding the applications of BNCT.

### SUMMARY OF THE INVENTION

In view of the above problems, the present disclosure provides a radiotherapy system including a neutron acceptor, an aerosolization device, and an energy beam generator. The aerosolization device is used to aerosolize the neutron acceptor.

The present disclosure further provides a method for treating cancer, including: administering to a subject in need thereof an effective amount of an aerosolized neutron acceptor, and irradiating the subject with neutrons.

The present disclosure also provides a method for diagnosing cancer, including: administering to a subject in need thereof an effective amount of an aerosolized radioactive agent; and receiving an energy beam signal from the subject.

The present disclosure also provides a method for delivering a neutron acceptor to a subject during radiotherapy, including: aerosolizing the neutron acceptor; and administering an effective amount of the neutron acceptor to the subject. The neutron acceptor includes an element that can be triggered or activated by neutron irradiation.

The present disclosure can significantly enhance the T/N ratio of the neutron acceptors in the subject by aerosol administration to the subject, so as to improve the effects of radiation therapy or diagnosis, and avoid damage to the healthy cells.

The present disclosure will be more readily understandable by reference to the following descriptions and drawings.

### BRIEF DESCRIPTION OF THE DRAWINGS

FIG. 1 shows a lung-tumor animal model in accordance with at least one embodiment of the present disclosure.

FIG. 2 illustrates a boron-phenylalanine-fructose complex (BPA-Fr) delivery process in accordance with at least one embodiment of the present disclosure.

FIG. 3A shows boron-10 concentrations in tumor tissue and normal tissue 5, 10, and 15 minutes after inhalation administration of aerosolized BPA-Fr in tumor-bearing and non-tumor-bearing (normal) mice.

FIG. 3B shows boron-10 uptake (tumor/normal tissue ratio) without BPA-Fr administration (Sham), 15 minutes after intraperitoneal injection (IP 15), 5 minutes after inhalation administration (Inhale 5), 10 minutes after inhalation administration (Inhale 10), and 15 minutes after inhalation administration (Inhale 15) in mice.

FIG. 3C shows boron-10 concentrations in tumor tissue and blood 5, 10, and 15 minutes after inhalation administration of aerosolized BPA-Fr in tumor-bearing mice and non-tumor-bearing (normal) mice.

FIG. 3D shows boron-10 uptake (tumor tissue/blood ratio) without BPA-Fr administration (Sham), 15 minutes after intraperitoneal injection (IP 15), 5 minutes after inhalation administration (Inhale 5), 10 minutes after inhalation administration (Inhale 10), and 15 minutes after inhalation administration (Inhale 15) in mice.

### DETAILED DESCRIPTION

The following embodiments are provided to illustrate the present disclosure in detail. A person having ordinary skill in the art can easily understand the advantages and effects of the present disclosure after reading the disclosure of this specification and can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other elements or methods disclosed in any embodiments of the present disclosure.

As used herein, the article “a,” “an,” and “the” are intended to one or more than one (i.e., at least one) grammatical object of the article, unless the context clearly indicates otherwise, and the term “or” is used interchangeably with the term “and/or” unless the context clearly indicates otherwise.

As used herein, the phrase “at least one,” in reference to a list of one or more elements, should be understood to mean at least one element selected from any one or more of the elements in the list of elements, but not necessarily including at least one of each element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the terms “including,” “comprising,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

The numeral ranges used herein are inclusive and combinable, any numeral value that falls within the numeral scope herein could be taken as a maximum or minimum value to derive the sub-ranges therefrom. For example, the numeral range “from 10 ppm to 200 ppm” comprises any sub-ranges between the minimum value of 10 ppm to the maximum value of 200 ppm, such as the sub-ranges from 10 ppm to 50 ppm, from 150 ppm to 200 ppm, from 30 ppm to 80 ppm, and so on. In addition, a plurality of numeral values used herein can be optionally selected as maximum and minimum values to derive numerical ranges. For instance, the numerical ranges of 20 ppm to 40 ppm, 20 ppm to 60 ppm, and 40 ppm to 60 ppm can be derived from the numeral values of 20 ppm, 40 ppm, and 60 ppm.

As used herein, the term “about” generally refers to the numerical value meant to encompass variations of +20%, +10%, +5%, +1%, +0.5%, or +0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the like disclosed herein should be understood as modified in all instances by the term “about.”

As used herein, the term “treat,” “treating,” or “treatment” refers to obtaining a desired pharmacologic or physiologic effect, e.g., partially or completely preventing, ameliorating, mitigating or managing a disorder, a symptom or a condition associated with a disease. The term “treat,” “treating,” or “treatment” as used herein may refer to the application or administration of at least one therapeutic agent or approach to a subject having a disorder, a symptom, or a condition associated with a disease, with the purpose to partially or completely ameliorate, alleviate, relieve, delay onset of, reduce the severity of, inhibit the progression of, or reduce the incidence of at least one disorder, symptom, or condition associated with the disease. In some embodiments, treatment may be administered to a subject exhibiting only an early sign of such disorders, symptoms, or conditions associated with a disease for the purpose of decreasing the risk of developing the same.

As used herein, the term “diagnose,” “diagnosing,” or “diagnosis,” refers to the classification or identification of the molecular or pathological disease or condition. In some embodiments, the term “diagnose,” “diagnosing,” or “diagnosis” may refer to the identification of a specific type of cancer, e.g., but not limited to lung cancer.

As used herein, the terms “subject” and “patient” are interchangeable and refer to an animal, e.g., a mammal. The term “subject” is intended to refer to both the male and female gender unless one gender is specifically indicated. In at least one embodiment of the present disclosure, the subject is a rodent, a murine, a monkey, a dog, a cat, a cow, a horse, an emu, a sheep, a deer, a wolf, a fox, a pig, a rabbit, a chicken, an ostrich, or a human, but the present disclosure is not limited thereto. In some embodiments, the subject is a human.

As used herein, the term “effective amount,” refers to the amount of an active agent or a pharmaceutical composition that is sufficient to treat, prevent, or diagnose the specified disorder, a symptom, or a condition associated with a disease in a subject in need thereof. In some embodiments, the effective amount may vary by a person ordinarily skilled in the art, depending on excipient usage, routes of administration, the possibility of co-usage with other therapeutic treatments, and the condition to be treated.

As used herein, the term “administer,” “administering” or “administration” refers to the placement of an active ingredient into a subject by a method or route that results in at least partial localization of the active ingredient at a desired site to produce the desired effect. For example, the active ingredient of the present disclosure may be administered to the subject by inhalation, but the present disclosure is not limited thereto.

As used herein, the term “pharmaceutically acceptable carrier” refers to a pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexing agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject (e.g., but not limited to a mammal) without excessive toxicity, allergic response, irritation, immunogenicity, or other complications or problems. See, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

At least one embodiment of the present disclosure provides a method for treating cancer, including: administering to a subject in need thereof an effective amount of an aerosolized neutron acceptor; and irradiating the subject by neutrons. At least one embodiment of the present disclosure provides a pharmaceutical composition for use in treating cancer in a subject in need thereof, which contains an effective amount of an aerosolized neutron acceptor and a pharmaceutically acceptable carrier, and the subject is irradiated by neutrons.

At least one embodiment of the present disclosure provides a method for diagnosing cancer, including: administering to a subject in need thereof an effective amount of a radioactive agent; and receiving an energy beam signal from the subject. At least one embodiment of the present disclosure provides a pharmaceutical composition for use in diagnosing cancer in a subject in need thereof, which contains an effective amount of an aerosolized radioactive agent and a pharmaceutically acceptable carrier thereof.

In at least one embodiment of the present disclosure, the cancer is a lung cancer. In some embodiments, the lung cancer may be selected from the group consisting of non-small cell lung cancer, small cell lung cancer, a Pancoast tumor, a bronchial tumor, pleuropulmonary blastomas, a lung carcinoid tumor, and any combination thereof, but the present disclosure is not limited thereto. In some embodiments, the small cell lung cancer may be selected from the group consisting of adenocarcinoma, squamous cell carcinoma, large cell carcinoma, and any combination thereof, but the present disclosure is not limited thereto.

In at least one embodiment of the present disclosure, the neutron acceptor may include an element that can be triggered or activated by neutron irradiation. In some embodiments, the neutron acceptor may be labeled by a radionuclide. In some embodiments, the neutron acceptor may be a boron source or a gadolinium source. In some embodiments, the boron source may be at least one selected from the group consisting of boronophenylalanine, sodium borocaptate, ammonia-carboxy-borane, sodium borate, borane salt, copper tetracarboranyltetraphenylporphyrin, o-carboranylalanine, boron-rich porphyrin conjugate, boron-containing nucleoside, boron-containing oligonucleotid, boron antibody conjugate, boron containing thiouracil derivative, boron containing methionine analogue, boronated pyridine base, boronated purine base, and derivative or isomer thereof, but the present disclosure is not limited thereto. In some embodiments, the boron source may be at least one selected from the group consisting of a boronophenylalanine-fructose complex (BPA-Fr), boronophenylalanine, para-boronophenyalanine, meta-boronophenyalanine, ortho-boronophenyalanine, ¹⁸F-labeled boronophenyalanine, ¹⁸F-labeled para-boronophenyalanine, ¹⁸F-labeled meta-boronophenyalanine, ¹⁸F-labeled ortho-boronophenyalanine, boronophenylalanine-amide alkyl dodecaborate, and amide of boronophenylalanine, but the present disclosure is not limited thereto.

In at least one embodiment of the present disclosure, the radioactive agent may include a radionuclide selected from the group consisting of ¹⁸F, ¹⁵O, ¹³N, ¹¹C, ⁶⁷Ga, ^{99m}Tc, ¹²³I, ¹²⁴I, ¹²⁵I, ¹³¹I, ²⁰¹Tl, and ¹¹¹In. In at least one embodiment of the present disclosure, the radioactive agent may include a neutron acceptor labeled by a radionuclide, e.g., the radioactive agent may include a radionuclide-labeled boron source or a radionuclide-labeled gadolinium source, but the present disclosure is not limited thereto.

In at least one embodiment of the present disclosure, the effective amount may be from about 10 ppm of blood concentration to about 200 ppm of blood concentration, e.g., about 20 ppm to about 40 ppm of blood concentration, about 50 ppm to about 170 ppm of blood concentration, or about 60 ppm to about 100 ppm of blood concentration, but the present disclosure is not limited thereto. In some embodiments, the effective amount may be about 10 ppm, 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm, 160 ppm, 170 ppm, 180 ppm, 190 ppm, or 200 ppm of blood concentration, but the present disclosure is not limited thereto.

In at least one embodiment of the present disclosure, the effective amount may be from about 0.1 mg/kg to about 500 mg/kg, e.g., about 0.2 mg/kg to about 1 mg/kg, about 10 mg/kg to about 50 mg/kg, about 100 mg/kg to about 200 mg/kg, or about 300 mg/kg to about 400 mg/kg, but the present disclosure is not limited thereto. In some embodiments, the effective amount may be about 0.1 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 200 mg/kg, 250 mg/kg, 300 mg/kg, 350 mg/kg, 400 mg/kg, 450 mg/kg, or 500 mg/kg, but the present disclosure is not limited thereto.

In at least one embodiment of the present disclosure, the energy beam generator may be a neutron beam generator.

In at least one embodiment of the present disclosure, the aerosolization device may be a pharmaceutically acceptable atomizer, a nebulizer, an inhaler, or a compressor, but the present disclosure is not limited thereto. In some embodiments, the nebulizer may be a vibrating mesh nebulizer (VMN).

In at least one embodiment of the present disclosure, the neutron acceptor may be administered to a respiratory system of the subject. In some embodiments, the respiratory system may include a respiratory tract or a lung, but the present disclosure is not limited thereto. In some embodiments, the respiratory system may include an oral cavity, nasal cavity, pharynx, larynx, trachea, carina, primary bronchi, secondary bronchus, tertiary bronchi, bronchioles, alveoli, or any combination thereof, but the present disclosure is not limited thereto.

In at least one embodiment of the present disclosure, the neutron acceptor neutron acceptor may be administered by inhalation.

In at least one embodiment of the present disclosure, the administration time may be about 1 to 20 minutes, e.g., but not limited to about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, or 20 minutes.

In some embodiments, the inhalation administration time may be about 5 to 10 minutes, 6 to 8 minutes, or 2 to 5 minutes, but the present disclosure is not limited thereto.

In at least one embodiment of the present disclosure, the neutron acceptor may be aerosolized by a pharmaceutically acceptable atomizer, a nebulizer, an inhaler, or a compressor.

In at least one embodiment of the present disclosure, the subject may suffer from a lung cancer.

In at least one embodiment of the present disclosure, the method for delivering a neutron acceptor to a subject during radiotherapy further contains irradiating the subject by neutrons.

In at least one embodiment of the present disclosure, the aerosolized neutron acceptor may be administered to the respiratory system of the subject by inhalation.

In at least one embodiment of the present disclosure, the radiotherapy may be a neutron capture therapy. In some embodiments, the neutron capture therapy may be a boron neutron capture therapy.

In at least one embodiment of the present disclosure, compared to other administration methods or dosage forms, the administration of the aerosolized neutron acceptor or the aerosolized radioactive agent to a subject can significantly increase the T/N ratio of the neutron acceptor in the subject and achieve relatively high accumulation of neutron acceptor in tumor and relatively low accumulation of neutron acceptor in normal cells or blood within a very short period (e.g., but not limited to 5 to 10 minutes) to improve the effect of treatment or diagnosis and avoid damage to healthy cells. In some embodiments, a preferred mode of administration is inhalation administration, e.g., inhaling aerosolized neutron acceptor or aerosolized radioactive agent through the mouth or nose of the subject, but the present disclosure is not limited thereto.

In at least one embodiment of the present disclosure, the present disclosure shows a much higher T/N ratio of the boron source in the subject as compared with the minimum clinical treatment requirement of a T/N ratio. Hence, the present disclosure can significantly enhance the T/N ratio of the boron source in the subject to improve the therapeutic effect of BNCT and avoid damage to healthy cells. However, the present disclosure is not limited to boron sources. In other embodiments, other neutron acceptors and neutron capture therapies also have similar excellent effects.

The following are Examples further demonstrating the effect of the present disclosure, but not limiting the scope of the present disclosure.

### Examples

The present disclosure is further described by means of the following examples. However, these examples are only illustrative of the disclosure, and in no way limit the scope and meaning of the present disclosure. Actually, many modifications and variations of the present disclosure will be apparent to those skilled in the art upon reading the present disclosure, and can be made without departing from its scope.

### Materials and Methods

### Lung-Tumor Animal Models

### Animal

Male BALB/c nude mice aged 6 to 8 weeks (average weight 25 grams) were used. These mice were raised in a pathogen-free environment at a temperature of 21±2° C. and a relative humidity of 30% to 70% at the animal center. Specialized personnel were responsible for their feeding.

### Tumor Formation

The A549 (adenocarcinoma) cell lines were maintained in Ham's F-12K medium containing 10% FBS (90% Ham's F-12K and 10% FBS) and 1% penicillin. A549 (10⁴/1 μl) cells were harvested and suspended in Ham's F-12K. Before injection, the cells were mixed with matrigel at a 1:1 ratio and kept on ice until injection. For injection, the cell mixture was gently mixed and transferred into a 30-μl syringe fitted with a 30-gauge needle. The syringe was loaded on the stereotactic injection system, and 20 μl of cell mixture was injected into each mouse.

Mice were anesthetized using isoflurane and the fur covering the left thorax was shaved. The mice were then positioned in the lateral decubitus position with the left chest facing up and a small (0.5 to 1 cm) incision was made over the skin just below the scapula. Next, the chest wall muscles were gently spread until the intercostal space and pleura were clearly visible. Then, the ribs were counted from the lower border of the rib cage upward, and the site of tumor injection between the fourth and fifth ribs at the posterior axillary line was determined. At this stage, the syringe was gently advanced until its tip finely touched the intercostal space. The syringe was then advanced 3.5 to 4.0 mm into the lung parenchyma and the tumor cell mixture was injected. After injection of the cells, the syringe was withdrawn from the tissue, and the covering muscles and skin were closed.

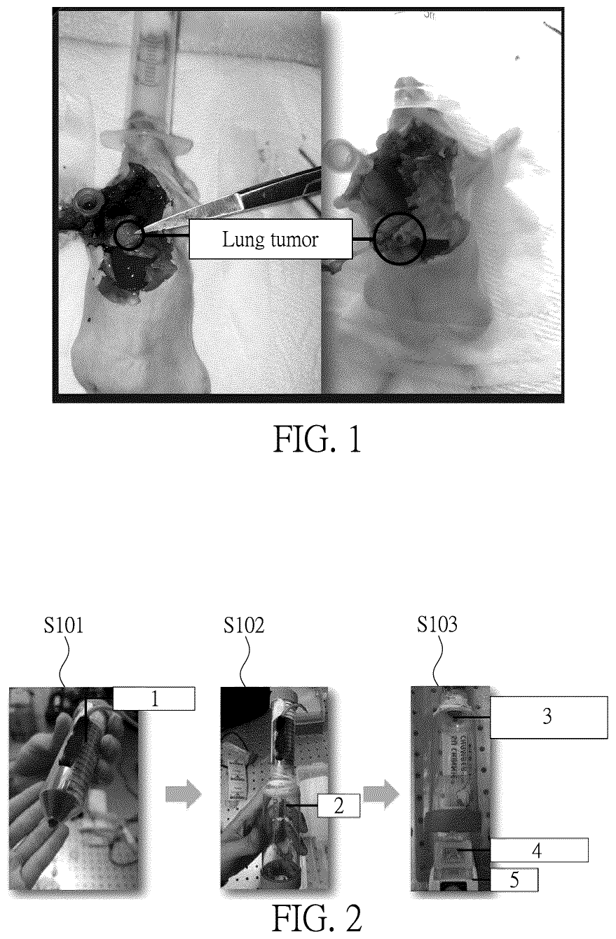

As shown in FIG. 1, two weeks after the injection of A549 cells, the tumor growth in the left lung (circle) can be seen when the chest was opened.

### Aerosolized BPA Delivery

The boron-phenylalanine-fructose complex (BPA-Fr) was drawn out in an appropriate volume using a 5 mL syringe, and then filtered through a 13-mm Acrodisc syringe filter containing a 0.2 μm supor membrane (Pall Life Sciences, Ann Arbor, USA) to remove any ice crystals present in the liquid medication.

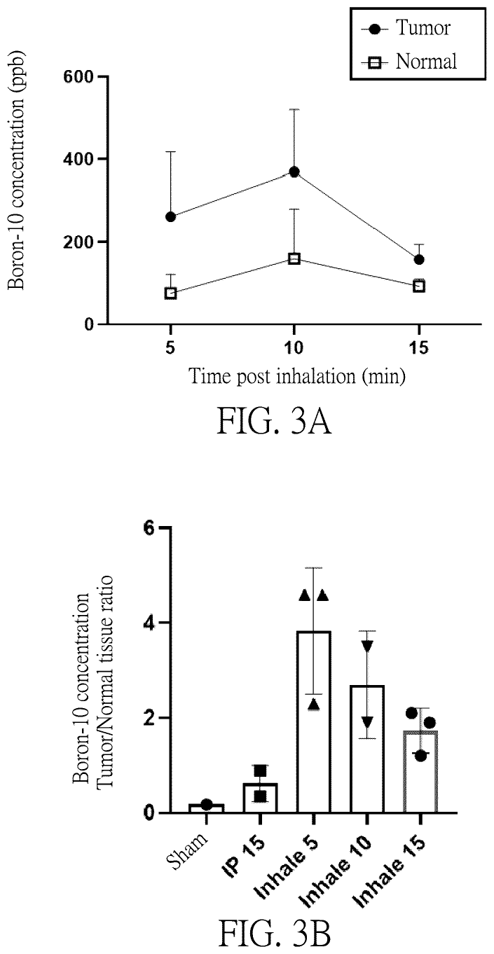

As shown in step S101 of FIG. 2, after the mice had been anesthetized with isoflurane, the mice were placed in a specially designed container 1, e.g., a restraint chamber, with only their noses exposed within the Aerochamber. As shown in step S102 of FIG. 2, the container 1 was connected to a holder 2 (e.g., but not limited to a valve holding chamber (VHC)), and the junction of the container 1 and the holder 2 was sealed with a single-layer sealing film. The holder 2 was connected to an aerosolization device 5 (e.g., but not limited to a vibrating mesh nebulizer (VMN)) with a BPA-Fr drug 4 shown in step S103 of FIG. 2 to effectively prevent aerosol backflow, and expose the mice nasal cavity 3.

The calculated dose of filtered BPA-Fr, at a concentration of 13 μL per gram, was placed into the medicine cup of the VMN. The power was then turned on, and the medication was manually administered for five seconds followed by a 30-second pause to ensure sufficient time for the mice to inhale the medication. The inhalation treatment was considered complete when the medication had been fully administered and no more mist was coming out of the device. Then, the medication cup was filled with 100 μL of phosphate buffered saline and continued to administer aerosol for the mice to inhale, in order to reduce residual drugs in the cup.

Analyze BPA Concentration with Mass Spectroscopy

Boron compound administration protocols were assessed in 2 mice, 2 weeks post-injection of 10⁵ A549 cells. The BPA-Fr was administered via inhalation at a dose of 0.3 mg BPA/g bodyweight.

Two hours after the administration of the boron compounds, the animals were then sacrificed by overdose of anesthesia immediately prior to tissue sampling. The blood samples were collected by cardiac puncture. The right lung of each mouse was collected as normal tissue, while the left lung, which exhibited visible tumor nodules, was excised and collected. The collected tissues and blood samples were dissolved in 10% nitric acid for subsequent measurement of boron-10 using ICP-MS.

### Result

In the high-dose drug administration group of the present disclosure, the inhalation of 0.3 mg/g of BPA-Fr was administered to BALB/c nude mice with lung tumors. The BPA-Fr was inhaled for approximately 6 to 8 minutes and allowed to be absorbed in the mice's bodies for 2 hours. Subsequently, the concentration of 1B was measured using ICP-MS (Thermo Fisher Scientific iCAP TQ). The concentration of 1B in the lung tumor tissue was 1782.96 ppm, whereas in the normal lung tissue, it was 51.42 ppm. The T/N ratio was calculated to be 34.67, indicating that it satisfies the minimum clinical treatment requirement of a T/N ratio greater than 2.5, as shown in Table 1. Therefore, compared to the minimum clinical treatment requirement of a T/N ratio, the present disclosure can bring about an effect on promoting the boron source to have a higher T/N ratio in the subject.

TABLE 1
Boron-10 uptake into lung and normal lung tissue and
T/N ratio by the high dose inhalation administration
method (Clinical standard for BPA-Fr: T/N ratio > 2.5)

| Experiment group | High-dose group - test 1 |
|---|---|
| Boron-10 concentration accumulated in | 1782.96 |
| lung tumor (ppm) | |
| Boron-10 concentration accumulated in | 51.42 |
| normal lung tissue (ppm) | |
| T/N ratio | 34.67 |

*T/N ratio (Boron-10) = lung tumor (ppm)/normal lung tissue (ppm)

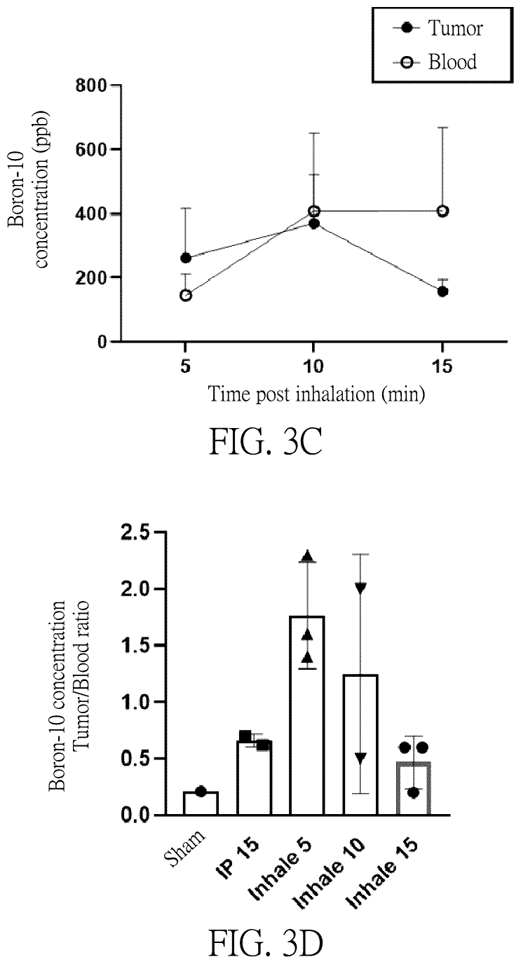

In addition, FIG. 3B and FIG. 3D show that compared with the Sham and intraperitoneal injection, inhalation administration can uptake aerosolized BPA-Fr faster; or, under the same time, the uptake rate of aerosolized BPA-Fr is better than that of intraperitoneal injection.

In terms of inhalation administration, sedimentation in the alveoli of the drug occurs after a living subject inhales the drug from the lung. The microvessel on the alveoli absorbs the drug, which then enters the tumor or healthy normal cells in the lung, gradually enters the blood vessel at the same time, and finally circulates throughout the body's blood. The BPA-Fr drug will eventually be converted into boron-10 for measurement, and the results represent level of the drug in cells or blood. In aspect of cancer treatment or diagnosis, it is better that the more boron-10 accumulates in the tumor and the less boron-10 accumulates in normal cells or blood. FIG. 3A shows that the boron-10 concentration in tumor cells and normal cells changes over time after the living subject inhales the same dose of aerosolized BPA-Fr drug. FIG. 3C shows the boron-10 concentration in tumor cells and blood cells changes over time after the living subject inhales the same dose of aerosolized BPA-Fr drug. It can be seen from FIG. 3A and FIG. 3C that relatively high boron-10 accumulation in tumors and relatively low boron-10 accumulation in normal cells or blood can be achieved within a short time (5 to 10 minutes) after the living subject inhales the aerosolized BPA-Fr drug, which shows the advantages of the present disclosure in therapeutic or diagnostic applications.

The above descriptions are only the preferred embodiments of the present disclosure. All equivalent changes and modifications made in accordance with the claims of the present disclosure shall fall within the scope of the present invention.

What is claimed is:

1. A radiotherapy system, comprising:
a neutron acceptor;
an aerosolization device for aerosolizing the neutron source; and
an energy beam generator.

2. The radiotherapy system of claim 1, wherein the energy beam generator is a neutron beam generator.

3. The radiotherapy system of claim 1, wherein the neutron acceptor comprises an element that can be triggered or activated by neutron irradiation.

4. The radiotherapy system of claim 1, wherein the neutron acceptor is labeled by a radionuclide.

5. The radiotherapy system of claim 4, wherein the neutron acceptor is a boron source or a gadolinium source, and the radionuclide is at least one selected from the group consisting of ¹⁸F, ¹⁵O, ¹³N, ¹¹C, ⁶⁷Ga, ^{99m}Tc, ¹²³I, ¹²⁴I, ¹²⁵I, ¹³¹I, ²⁰¹Tl, and ¹¹¹In.

6. A method for treating cancer, comprising:
administering to a subject in need thereof an effective amount of an aerosolized neutron acceptor; and
irradiating the subject with neutrons.

7. The method of claim 6, wherein the cancer is lung cancer.

8. The method of claim 6, wherein the neutron acceptor comprises an element that can be triggered or activated by neutron irradiation.

9. The method of claim 6, wherein the neutron acceptor is labeled by a radionuclide.

10. The method of claim 6, wherein the neutron acceptor is a boron source or a gadolinium source, and the radionuclide is at least one selected from the group consisting of ¹⁸F, ¹⁵O, ¹³N, ¹¹C, ⁶⁷Ga, ^{99m}Tc, ¹²³I, ¹²⁴I, ¹²⁵I, ¹³¹I, ²⁰¹Tl, and ¹¹¹In.

11. A method for diagnosing cancer, comprising:
administering to a subject in need thereof an effective amount of an aerosolized radioactive agent; and
receiving an energy beam signal from the subject.

12. The method of claim 11, wherein the cancer is a lung cancer.

13. The method of claim 11, wherein the radioactive agent comprises at least one radionuclide selected from the group consisting of ¹⁸F, ¹⁵O, ¹³N, ¹¹C, ⁶⁷Ga, ^{99m}Tc, ¹²³I, ¹²⁴I, ¹²⁵I, ¹³¹I, ²⁰¹Tl, and ¹¹¹In.

14. The method of claim 11, wherein the radioactive agent comprises a neutron acceptor labeled by a radionuclide.

15. The method of claim 14, wherein the neutron acceptor comprises an element that can be triggered or activated by neutron irradiation.

16. A method for delivering a neutron acceptor to a subject during radiotherapy, comprising:
aerosolizing the neutron acceptor; and
administering an effective amount of the neutron acceptor to the subject.

17. The method of claim 16, further comprising irradiating the subject by neutrons.

18. The method of claim 16, wherein the aerosolized neutron acceptor is administered to a respiratory system of the subject by inhalation.

19. The method of claim 16, wherein the subject suffers from a lung cancer.

20. The method of claim 16, wherein the radiotherapy is a neutron capture therapy.
